(19) United States
(12) Patent Application Publication
(10) Pub. No.: US 2018/0272049 A1
(43) Pub. Date: Sep. 27, 2018

(54) BLOOD RECIRCULATION DEVICE

(71) Applicant: CATHOLIC KWANDONG UNIVERSITY INDUSTRY FOUNDATION, Gangwon-do (KR)

(72) Inventor: Cheesoon YOON, Daejeon (KR)

(21) Appl. No.: 15/546,439

(22) PCT Filed: Jan. 26, 2015
(86) PCT No.: PCT/KR2015/000805

§ 371 Date: Jul. 26, 2017

(51) Int. Cl. A61M 1/00; A61M 1/02; A61M 1/36

(52) U.S. Cl. CPC A61M 1/0007; A61M 1/0259; A61M 1/3666; A61M 1/0023; A61M 2202/0413; A61M 2205/3331; A61M 2205/3389; A61M 2205/3393; A61M 2205/07; A61M 1/0094

(57) ABSTRACT

The present invention relates to a blood recirculation device. More particularly, the present invention relates to a blood recirculation device capable of continuous blood suction and capable of minimizing mechanical damage to the blood during such a process. The blood recirculation device according to the present invention repeats suction with one side unit and repeats a process of transferring stored blood with the other side unit, thereby being capable of continuous blood suction and capable of efficiently supplying suctioned blood to an erythrocyte washing system of an autologous blood transfusion machine or a cardiopulmonary bypass machine.

### TECHNICAL FIELD

The present invention relates to a blood recirculation device. More specifically, the present invention relates to a blood recirculation device capable of achieving continuous suction of blood and minimizing mechanical damage to blood during such a procedure.

### BACKGROUND ART

For the reuse of blood from bleeding occurring during surgery, there is a method in which the blood from bleeding is sucked using a roller pump, stored in a reserve chamber of an artificial heart-lung machine for a short time, and then again supplied to a venous line of the artificial heart-lung machine, in the case of surgery using heparin, such as heart surgery.

This method is advantageous in that the total circulating blood volume can be maintained by immediately supplying the blood from bleeding to the artificial heart-lung machine, but is disadvantageous in that mechanical damage to blood by a roller pump is caused by using the roller pump as a unit for transferring the blood from bleeding.

Currently, a centrifugal pump is frequently used for an artificial heart-lung machine, and the reason is that the centrifugal pump, compared with a roller pump, causes less blood damage during the driving of the artificial heart-lung machine. However, the centrifugal pump does not work with a mixture including air, and therefore, the centrifugal pump is used mainly to circulate the blood in arterial and venous lines of the artificial heart-lung machine and the roller pump is used for the blood in other lines. The principle of the roller pump is that a flexible tube is continuously pressed to generate negative pressure therein and thus to suck blood. Here, the pressure on the tube results in damage to the blood in the tube, and the administration of the damaged blood may cause various complications.

On the other hand, blood from bleeding occurring during surgery needs to be promptly removed because the blood may interfere with the surgical field of vision, and therefore, the suction function needs to be continuously conducted. In addition, in cases where the sucked blood is recirculated, the less the blood is damaged, the lower the risk of complications.

Therefore, the use of the roller pump needs to be avoided in order to allow continuous suction while attaining less blood damage and to minimize blood damage. For the suction of blood without using a roller pump, negative pressure may be used, but the negative pressure is difficult to maintain while the collected blood is injected into the artificial heart-lung machine, and continuous suction cannot be used.

Throughout the entire specification, many papers and patent documents are referenced and their citations are represented. The disclosures of the cited papers and patent documents are entirely incorporated by reference into the present specification, and the level of the technical field within which the present invention falls and the details of the present invention are thus explained more clearly.

### DETAILED DESCRIPTION

### Technical Problem

Under this background, the present inventors have endeavored to develop a device capable of achieving continuous suction while preventing mechanical damage to sucked blood during the use of a roller pump. As a result, the present inventors have developed a blood recirculation device wherein a plurality of device units, each of which sucks blood using negative pressure and then automatically transfers the blood to an artificial heart-lung machine when the volume of the sucked blood arrives at a certain level, are installed, so that while the blood stored in one device unit is transferred to the artificial heart-lung machine, the blood from bleeding can be sucked into another device unit, thereby achieving continuous suction and preventing mechanical damage to blood.

Accordingly, an aspect of the present invention is to provide a blood recirculation device.

Other purposes and advantages of the present invention will become more obvious with the following detailed description of the invention, claims, and drawings.

### Technical Solution

In accordance with an aspect of the present invention, there is provided a blood recirculation device, including: first and second chambers each including a blood storage tank in which the blood sucked from a subject is stored, an atmospheric pressure/positive pressure supply part provided in one surface of the blood storage tank to supply atmospheric pressure or positive pressure to the blood storage tank by opening of an opening/closing control element, and an opening/closing control part for sensing the amount of the blood stored in the blood storage tank to control opening or closing of a plurality of opening/closing control elements installed in the device; first and second negative pressure supply pipes extending from surfaces of the blood storage tanks of the first and second chambers, respectively, ends of the first and second negative pressure supply pipes being connected to a negative pressure supply apparatus, wherein the first and second negative pressure supply pipes supply negative pressure to the blood storage tanks of the first and second chambers by opening of the opening/closing control elements provided therein, respectively; first and second blood supply pipes extending from surfaces of the blood storage tanks of the first and second chambers, respectively, wherein the first and second blood supply pipes supply the sucked blood to the blood storage tanks of the first and second chambers by opening of the opening/closing control elements provided therein, respectively; and first and second blood transfer pipes extending from surfaces of the blood storage tanks of the first and second chambers, respectively, wherein the first and second blood transfer pipes discharge the blood, which is stored in the blood storage tanks of the first and second chambers, by opening of the opening/closing control elements provided therein, respectively.

The present inventors have endeavored to develop a device capable of achieving continuous suction while preventing mechanical damage to sucked blood during the use of a roller pump. As a result, the present inventors have developed a blood recirculation device wherein a plurality of device units, each of which sucks blood using negative pressure and then automatically transfers the blood to an artificial heart-lung machine when the volume of the sucked blood arrives at a certain level, are installed, so that while the blood stored in one device unit is transferred to the artificial heart-lung machine, the blood from bleeding can be sucked into another device unit, thereby achieving continuous suction and preventing mechanical damage to blood.

According to an embodiment of the present invention, the opening/closing control part may include at least one sensor selected from the group consisting of a level sensor, a pressure sensor, and a weight sensor.

According to an embodiment of the present invention, the first and second negative pressure supply pipes may extend from upper surfaces of the blood storage tanks of the first and second chambers, respectively.

According to an embodiment of the present invention, ones of the first and second negative pressure supply pipes may be connected to the same or different negative pressure supply apparatuses, respectively, or the first and second negative pressure pipes may be connected to a negative pressure supply apparatus by sharing a portion of a pipe.

According to an embodiment of the present invention, the first and second blood supply pipes may extend from upper surfaces of the blood storage tanks of the first and second chambers while being separated from the first and second negative pressure supply pipes, respectively.

According to an embodiment of the present invention, the first and second blood transfer pipes may extend from lower surfaces of the blood storage tanks of the first and second chambers, respectively.

According to an embodiment of the present invention, ones of the first and second blood transfer pipes may be connected to artificial heart-lung machines or cell savers, respectively, or the first and second blood transfer pipes may be connected to an artificial heart-lung machine or a cell saver by sharing a portion of a pipe.

According to an embodiment of the present invention, the blood stored in the blood storage tank of the second chamber may be discharged to the second blood transfer pipe while the sucked blood is supplied to the blood storage tank of the first chamber.

### Advantageous Effects

The features and advantages of the present invention are summarized as follows:

(i) The present invention provides a blood recirculation device.

(ii) The blood recirculation device of the present invention does not need to use a roller pump and thus can minimize mechanical damage to blood during a suction procedure, causes no risk of the rupture of a tube or the like because a tube of polyurethane or the like is not used in a roller pump, and can simplify and reduce the size of an artificial heart-lung machine because a roller pump is not used.

(iii) The blood recirculation device of the present invention can achieve continuous suction of blood and efficient supply of the sucked blood to an artificial heart-lung machine, or to a red blood cell (RBC) cleaning system of a cell saver, by repeating the suction using one unit and the transfer of the stored blood using another unit.

(iv) The blood recirculation device of the present invention can continuously perform a suction function and thus can frequently supply a small amount of blood to a red blood cell cleaning system of a cell saver, thereby achieving effective transfusion.

### BRIEF DESCRIPTION OF THE DRAWINGS

FIG. 1 is a diagram showing the structure of a blood recirculation device according to an embodiment of the present invention.

FIGS. 2 and 3 are diagrams showing the operation of the blood recirculation device according to an embodiment of the present invention when the device is applied to open heart surgery utilizing a cardiopulmonary bypass. FIG. 2 shows open/closed states of respective opening/closing control elements depending on the initial setup of the blood recirculation device; and FIG. 3 shows the flow of blood occurring in the blood recirculation device.

### MODE FOR CARRYING OUT THE INVENTION

Hereinafter, preferred embodiments of the present invention will be described in detail with reference to the accompanying drawings. However, the present invention is not restricted or limited to the embodiments. For reference, like numerals substantially refer to like elements throughout the present specification, and can be described with reference to contents described in other drawings in the following description, and the contents that are determined to be apparent to those skilled in the art or that are repeated may be omitted.

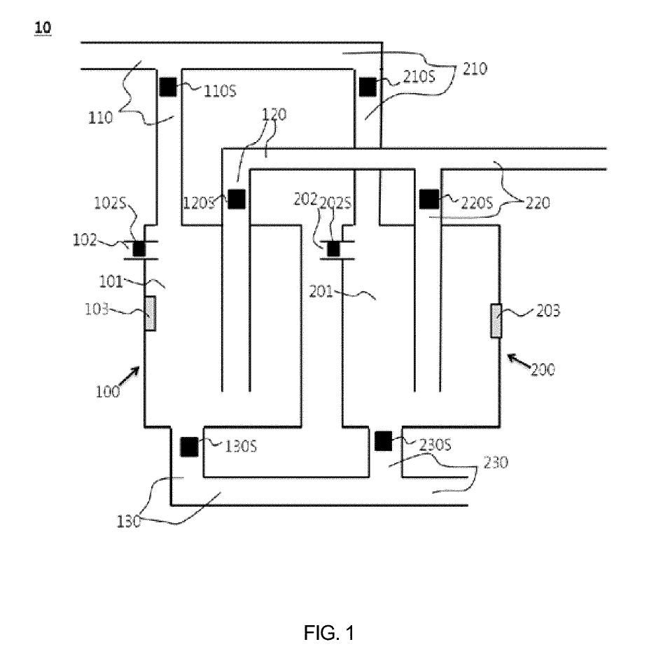

FIG. 1 is a diagram showing the structure of a blood recirculation device according to an embodiment of the present invention.

As shown in FIG. 1, a blood recirculation device 10 according to an embodiment of the present invention comprises first and second chambers 100 and 200, first and second negative pressure supply pipes 110 and 210, first and second blood supply pipes 120 and 220, and first and second blood transfer pipes 130 and 230, respectively.

In addition, the blood recirculation device 10 of the present invention may comprise, as needed, a plurality (three or more) of sets each consisting of a chamber, a negative pressure supply pipe, a blood supply pipe, and a blood transfer pipe.

The first and second chambers 100 and 200 have blood storage tanks 101 and 201, atmospheric pressure/positive pressure supply parts 102 and 202, and opening/closing control parts 103 and 203, respectively, and perform a function of storing the blood sucked from a subject and a function as an intermediary apparatus for recirculating the stored blood to the human body.

The blood storage tanks 101 and 201 are provided for temporarily storing the blood sucked from the subject. The shape and size of the blood storage tanks 101 and 201 are not limited, and the blood storage tanks 101 and 201 may be configured in an appropriate shape and size depending on the purpose for which the device of the present invention is used.

The atmospheric pressure/positive pressure supply parts 102 and 202 are provided in surfaces of the blood storage tanks 101 and 201 to supply atmospheric pressure or positive pressure to the blood storage tanks 101 and 201 by opening or closing of the opening/closing control elements 102S and 202S provided therein, respectively. Herein, the reference numeral of each of the opening/closing control elements is denoted in a manner such as, “the reference numeral of a component including each opening/closing control element +S”.

According to an aspect of the present invention, ends of the atmospheric pressure/positive pressure supply parts 102 and 202 are in contact with the outside, so that the atmosphere outside may flow into the blood storage tanks 101 and 201 by opening of the opening/closing control elements 102S and 202S, respectively.

According to another aspect of the present invention, ends of the atmospheric pressure/positive pressure supply parts 102 and 202 are connected with a positive pressure generation apparatus (not shown), so that the positive pressure can be supplied to the blood storage tanks 101 and 201 by opening of the opening/closing control elements 102S and 202S, respectively. For the positive pressure generation apparatus, a known positive pressure generation apparatus, such as a pump or a blower, may be used.

The opening/closing control parts 103 and 203 sense the amounts (volumes) of the blood, which flows into and is stored in the blood storage tanks 101 and 201, to perform a function of controlling the opening or closing of the plurality of opening/closing control elements (S) provided in the blood recirculation device 10, respectively. Through the functions of the opening/closing control parts 103 and 203, the amounts of the blood stored in the blood storage tanks 101 and 201 can be controlled and continuous suction and blood recirculation can be achieved.

The opening/closing control parts 103 and 203 can be configured as various sensors capable of sensing the amounts (volumes) of the blood flowing into the blood storage tanks 101 and 201.

According to an aspect of the present invention, the opening/closing control parts 103 and 203 include at least one sensor selected from the group consisting of a level sensor, a pressure sensor, and a weight sensor. The sensor generates an electric signal when sensing a predetermined value, and the generated electric signal is delivered to the respective opening/closing control elements S to control opening or closing of the opening/closing control elements S.

For example, pressure sensors may be attached on inside walls of the blood storage tanks 101 and 201 to recognize the amounts of blood stored in the blood storage tanks 101 and 201 through differences in pressure, respectively.

In another example, the level sensors recognize the changes in the surface height of the blood flowing into the blood storage tanks 101 and 201, so that the opening or closing of the opening/closing control elements S provided in the blood recirculation device 10 can be controlled. A method of operating a sensor by using an object floating on the blood to sense the height of the object, and a method of operating a sensor depending on the presence or absence of a light ray radiated when the height of the blood gathering in the blood storage tanks 101 and 201 is higher or lower than a predetermined height, or the like may be used.

The opening/closing control parts 103 and 203 may be configured as the same or different sensors.

The opening/closing control parts 103 and 203 may be configured as sensors recognizing different values even when the same sensors, such as a high-level sensor and a low-level sensor, or a high-voltage sensor or a low-voltage sensor, are used.

The opening/closing control parts 103 and 203 may include controllers for efficiently controlling the opening or closing of the plurality of opening/closing control elements S. These controllers receive electric signals from the sensors mounted on the respective chambers 100 and 200, and are then capable of controlling the opening or closing of the respective opening/closing control elements S depending on the programmed operations.

For example, the controllers can uniformly control the amounts per hour of blood flowing into the blood storage tanks 101 and 201. For example, the controllers compare the signal values received from the sensors mounted on the respective chambers 100 and 200 with predetermined values, and then control the inflowing amounts of blood by controlling the negative pressure or adjusting the inner diameters of the pipes installed in the device 10. Therefore, the blood recirculation device 10 of the present invention may include additional equipment (configuration) for allowing the controllers to perform the above functions.

For example, the additional equipment corresponds to a balloon installed in a pipe. The controllers can uniformly control the amount of blood flowing into the blood storage tanks 101 and 201 by controlling the pressures of the balloons to adjust the inner diameters of the pipes. An example of an element for controlling the pressure of the balloon may be an air injection/exhaustion element connected between a pipe connected to an end of the balloon and a pipe. The air injection/exhaustion element may be configured as an apparatus known in the art, such as an impeller or a pump.

The first and second negative pressure supply pipes 110 and 210 are passages in which air is movable, and perform a function of sucking the blood from bleeding during surgery to allow the blood to flow into the respective blood storage tanks 101 and 201 by supplying the negative pressure to the blood storage tanks 101 and 201 and the blood supply pipes 120 and 220 by opening of the opening/closing control elements 110S and 210S.

The first and second negative pressure supply pipes 110 and 210 extend from surfaces of the blood storage tanks 101 and 201, respectively, and ends of the extended portions are connected to a negative pressure supply apparatus. As the negative pressure supply apparatus, a known means capable of sucking the air in the blood recirculation device 10 to the outside may be used.

For example, the first and second negative pressure supply pipes 110 and 210 may be connected to a negative pressure pipe in an operating room.

According to an aspect of the present invention, the first and second negative pressure supply pipes 110 and 210 may extend from upper surfaces of the blood storage tanks 101 and 201 of the first and second chambers 100 and 200, respectively.

According to an aspect of the present invention, the first and second negative pressure supply pipes 110 and 210 may be connected to the same or different negative pressure supply apparatuses, respectively, or may be connected to a negative pressure supply apparatus by sharing a portion of a pipe as shown in FIG. 1.

According to an aspect of the present invention, the first and second negative pressure supply pipes 110 and 210 may include balloons therein, the balloons serving to control the flow of fluids. For example, one end of the balloon is connected to a pipe, of which one end is connected to the air injection/exhaustion element, and the negative pressure loaded inside the device 10 can be controlled by controlling the pressure inside the balloon through the air injection/exhaustion element.

The first and second blood supply pipes 120 and 220 are passages in which fluids (blood and air) move, and perform a function of sucking the blood from bleeding during surgery and moving the sucked blood to the blood storage tanks 101 and 201, by delivering the negative pressure in the respective blood storage tanks 101 and 201, which is supplied from the respective negative pressure supply pipes 110 and 210, to a suction tube by opening of the opening/closing control elements 120S and 220S provided in the first and second blood supply pipes 120 and 220.

According to an aspect of the present invention, the first and second blood supply pipes 120 and 220 may extend from upper surfaces of the blood storage tanks 101 and 201 while being separated from the first and second negative pressure supply pipes 110 and 210.

According to an aspect of the present invention, the first and second blood supply pipes 120 and 220 may be connected to a suction tube by sharing a portion of a pipe as shown in FIG. 1.

According to an aspect of the present invention, the first and second blood supply pipes 120 and 220 may include balloons therein, the balloons serving to control the flow of fluids. For example, one end of the balloon is connected to a pipe, of which one end is connected to the air injection/exhaustion element, and the amount of blood flowing into the blood storage tanks 101 and 201 can be controlled by controlling the pressure inside the balloon through the air injection/exhaustion element.

The first and second blood transfer pipes 130 and 230 function as passages in which the blood stored in the respective blood storage tanks 101 and 201 is discharged by opening of the opening/closing control elements 130S and 230S provided in the first and second blood transfer pipes 130 and 230.

According to an aspect of the present invention, the first and second blood transfer pipes 130 and 230 may extend from lower surfaces of the blood storage tanks 101 and 201, respectively. In this case, the discharge of the blood stored in the blood storage tanks 101 and 201 is attained by gravity and atmospheric pressure (or positive pressure).

According to an aspect of the present invention, ends of the first and second blood transfer pipes 130 and 230, which extend from surfaces of the blood storage tanks 101 and 201, may be connected to an artificial heart-lung machine or a cell saver, respectively. Alternatively, the first and second blood transfer pipes 130 and 230 may be connected to the above apparatus by sharing a portion of a pipe as shown in FIG. 1.

The plurality of opening/closing control elements S provided in the blood recirculation device 10 function to block the flow of fluids (air and/or blood). To achieve this purpose, the opening/closing control elements S may be configured by using a known opening/closing control element (e.g., a valve), without being limited thereto.

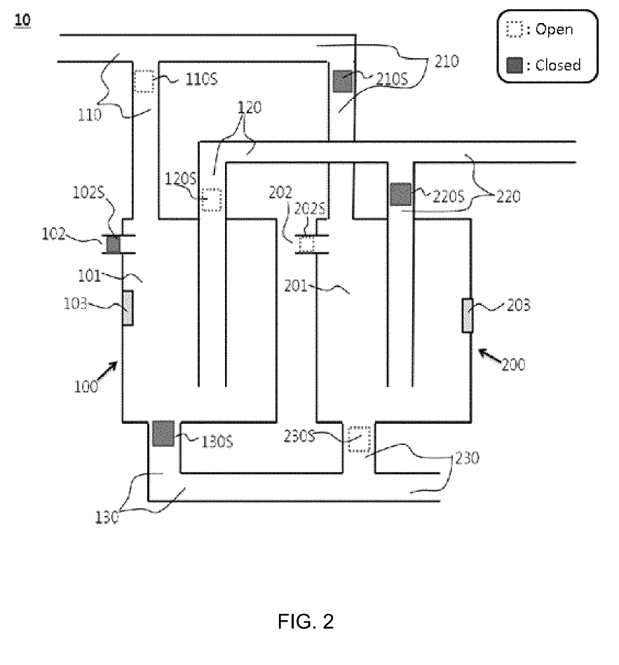

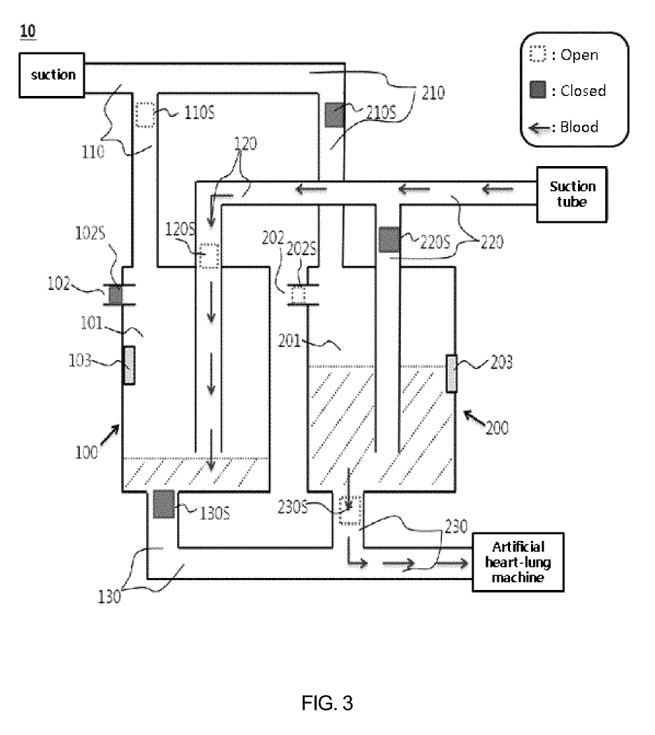

Hereinafter, the operation of the blood recirculation device according to an embodiment of the present invention will be described with reference to FIGS. 2 and 3.

FIGS. 2 and 3 are diagram showing the operation of the blood recirculation device according to an embodiment of the present invention when the device is applied to open heart surgery utilizing cardiopulmonary bypass. FIG. 2 shows the open and closed states of respective opening/closing control elements for the initial setup of the blood recirculation device, and FIG. 3 shows the flow of the blood in the blood recirculation device.

According to an aspect of the present invention, the blood recirculation device 10 may set the initial state shown in Table 1.

TABLE 1

| First chamber | | | | Second chamber | | | |
|---|---|---|---|---|---|---|---|
| 110S | 120S | 102S | 130S | 210S | 220S | 202S | 230S |
| OPEN | OPEN | CLOSED | CLOSED | CLOSED | CLOSED | OPEN | OPEN |

When the initially set blood recirculation device 10 is operated as shown in Table 1, the negative pressure is sequentially supplied to the blood storage tank of the first chamber 100 and the first blood supply pipe 120 by the first negative pressure supply pipe 110, of which one end is connected to a negative pressure supply apparatus, and thus, the blood from bleeding during surgery is sucked from a suction tube connected to one end of the first blood supply pipe 120, and the sucked blood starts to flow into the blood storage tank 101 of the first chamber 100 through the first blood supply pipe 120.

Thereafter, when the blood is stored in the blood storage tank 101 of the first chamber 100 to a predetermined amount (volume) and this condition is sensed by the opening/closing control part 103 installed in the blood storage tank 101, the opening/closing control elements 202S and 230S, which are provided in the atmospheric pressure/positive pressure supplying part 202 of the second chamber 200 and the second blood transfer pipe 230, are switched from the open state (opening) to a closed state (blocked) and the opening/closing control elements 120S and 220S, which are provided in the second negative pressure supply pipe 210 and the second blood supply pipe 220, are switched from the closed state to an open state, so that the negative pressure is supplied to the blood storage tank 201 of the second chamber 200 and the second blood supply pipe 220; and the opening/closing control elements 110S and 120S, which are provided in the first negative pressure supply pipe 110 and the first blood supply pipe 120, are switched from the open state to a closed state, so that the inflow of the blood into the blood storage tank 101 of the first chamber 100 is stopped, and the opening/closing control element 102S, which is provided in the atmospheric pressure/positive pressure supply part 102 of the first chamber 100, is switched from the closed state to an open state, so that atmospheric pressure or positive pressure is supplied to the blood storage tank 101 of the first chamber 100, and the opening/closing control element 130S, which is provided in the first blood transfer pipe 130, is also switched from the closed state to an open state.

Therefore, the blood is supplied to the blood storage tank 201 of the second chamber 200 through the second blood supply pipe 220, and the blood stored in the blood storage tank 101 of the first chamber 100 is transferred to a storage chamber of the artificial heart-lung machine (or a cell saver) through the first blood transfer pipe 130, and is again administered to a patient, thereby achieving blood recirculation.

Thereafter, when the blood is also stored in the blood storage tank 201 of the second chamber 200 to a predetermined amount (volume) and this condition is sensed by the opening/closing control part 203 installed in the blood storage tank 201, the opening/closing control elements 102S and 130S, which are provided in the atmospheric pressure/positive pressure supplying part 102 of the first chamber 100 and the first blood transfer pipe 130, are switched from the open state to a closed state, and the opening/closing control elements 110S and 120S, which are provided in the first negative pressure supply pipe 110 and the first blood supply pipe 120, are switched from the closed state to an open state, so that the blood flows into and is stored in the blood storage tank 101 of the first chamber 100; and the opening/closing control elements 210S and 220S, which are provided in the second negative pressure supply pipe 210 and the second blood supply pipe 220, are switched from the open state to a closed state, and the opening/closing control elements 202S and 230S, which are provided in the atmospheric pressure/positive pressure supply part 202 of the second chamber 200 and the second blood transfer pipe 230, are switched from the closed state to an open state, so that the blood stored in the blood storage tank 201 of the second chamber 200 is transferred to a storage chamber of the heart-lung machine (or a cell saver) through the second blood transfer pipe 230, and is again administered to the patient, thereby achieving blood recirculation.

As such, in the blood recirculation device 10 of the present invention, a procedure, in which the blood stored in the second chamber 200 is transferred to a device, such as an artificial heart-lung machine, while the blood sucked in the first chamber 100 is stored, and the blood stored in the first chamber 100 is transferred to a device, such as an artificial heart-lung machine, while the blood sucked in the second chamber 200 is stored, is repeatedly carried out, thereby achieving continuous suction and blood supply. Therefore, within the scope in which this procedure can be carried out, the opening or closing of the respective opening/closing control elements S can be simultaneously and sequentially performed.

### EXPLANATION OF REFERENCE SIGNS

10: blood recirculation device

100: first chamber

101: blood storage tank

102: atmospheric pressure/positive pressure supply part

103: opening/closing control part

110: first negative pressure supply pipe

120: first blood supply pipe

130: first blood transfer pipe

200: second chamber

201: blood storage tank

202: atmospheric pressure/positive pressure supply part

203: opening/closing control part

201: second negative pressure supply pipe

220: second blood supply pipe

230: second blood transfer pipe

S: opening/closing control unit

Although the present invention has been described in detail with reference to specific features thereof, it will be apparent to those skilled in the art that this description is only for a preferred embodiment and does not limit the scope of the present invention. Thus, the substantial scope of the present invention will be defined by the appended claims and equivalents thereof.

What is claimed is:

1. A blood recirculation device, comprising:
first and second chambers each comprising: a blood storage tank in which the blood sucked from a subject is stored; an atmospheric pressure/positive pressure supply part provided in one surface of the blood storage tank to supply atmospheric pressure or positive pressure to the blood storage tank by opening of an opening/closing control element; and an opening/closing control part for sensing the amount of the blood stored in the blood storage tank to control opening or closing of a plurality of opening/closing control elements installed in the device;
first and second negative pressure supply pipes extending from surfaces of the blood storage tanks of the first and second chambers, respectively, ends of the first and second negative pressure supply pipes being connected to a negative pressure supply apparatus, wherein the first and second negative pressure supply pipes supply negative pressure to the blood storage tanks of the first and second chambers by opening of the opening/closing control elements provided therein, respectively;
first and second blood supply pipes extending from surfaces of the blood storage tanks of the first and second chambers, respectively, wherein the first and second blood supply pipes supply the sucked blood to the blood storage tanks of the first and second chambers by opening of the opening/closing control elements provided therein, respectively; and
first and second blood transfer pipes extending from surfaces of the blood storage tanks of the first and second chambers, respectively, wherein the first and second blood transfer pipes discharge the blood, which is stored in the blood storage tanks of the first and second chambers, by opening of the opening/closing control elements provided therein, respectively.

2. The blood recirculation device of claim 1, wherein the opening/closing control part comprises at least one sensor selected from the group consisting of a level sensor, a pressure sensor, and a weight sensor.

3. The blood recirculation device of claim 1, wherein the first and second negative pressure supply pipes extend from upper surfaces of the blood storage tanks of the first and second chambers, respectively.

4. The blood recirculation device of claim 1, wherein ones of the first and second negative pressure supply pipes are connected to the same or different negative pressure supply apparatuses, respectively, or the first and second negative pressure pipes are connected to a negative pressure supply apparatus by sharing a portion of a pipe.

5. The blood recirculation device of claim 1, wherein the first and second blood supply pipes extend from upper surfaces of the blood storage tanks of the first and second chambers while being separated from the first and second negative pressure supply pipes, respectively.

6. The blood recirculation device of claim 1, wherein the first and second blood transfer pipes extend from lower surfaces of the blood storage tanks of the first and second chambers, respectively.

7. The blood recirculation device of claim 1, wherein ones of the first and second blood transfer pipes are connected to artificial heart-lung machines or cell savers, respectively, or the first and second blood transfer pipes are connected to an artificial heart-lung machine or a cell saver by sharing a portion of a pipe.

8. The blood recirculation device of claim 1, wherein the blood stored in the blood storage tank of the second chamber is discharged to the second blood transfer pipe while the sucked blood is supplied to the blood storage tank of the first chamber.
